(19) United States
(12) Patent Application Publication
(10) Pub. No.: US 2003/0060752 A1
(43) Pub. Date: Mar. 27, 2003

(54) Glaucoma device and methods thereof

(76) Inventors: Olav Bergheim, Laguna Hills, CA (US); Hosheng Tu, Newport Coast, CA (US); Morteza Gharib, San Marino, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/137,117

(22) Filed: May 1, 2002

Related U.S. Application Data
(63) Continuation-in-part of application No. 09/549,350, filed on Apr. 14, 2000.
(60) Provisional application No. 60/287,902, filed on May 1, 2001.

(51) Int. Cl. A61M 1/00

(52) U.S. Cl. 604/27

(57) ABSTRACT

A glaucoma treatment device for directing the flow of aqueous humor and reducing intraocular pressure for angle closure glaucoma is disclosed. The glaucoma device comprises an aqueous transporting element for transporting aqueous humor to bypass dysfunctional anatomical iris closure and restoring existing outflow pathways of the anatomical iris closure. The aqueous transporting element has an inlet end and an outlet end, wherein the inlet end is positioned inside an anterior chamber of an eye beyond an edge of the dysfunctional anatomic iris closure and the outlet end is positioned in proximity of trabecular meshwork of the eye. The device also serves to stent the space between the iris and an inner surface of a cornea of the eye.

### RELATED APPLICATIONS

This application is a continuation-in-part of U.S. patent application Ser. No. 09/549,350, filed Apr. 14, 2000, entitled “APPARATUS AND METHOD FOR TREATING GLAUCOMA,” and claims the benefit of U.S. Provisional Application No. 60/287,902, filed May 1, 2001, entitled “GLAUCOMA DEVICE AND METHODS THEREOF,” the entire contents of each one of which are hereby incorporated by reference herein.

### BACKGROUND OF THE INVENTION

1. Field of the Invention

The invention generally relates to medical devices and methods for reducing intraocular pressure in the animal eye and, more particularly, to the treatment of glaucoma by permitting aqueous humor to flow out of the anterior chamber through a surgically implanted pathway to restore existing outflow pathways.

2. Description of the Related Art

The human eye is a specialized sensory organ capable of light reception and able to receive visual images. The trabecular meshwork serves as a drainage channel and is located in the anterior chamber angle formed between the iris and the cornea. The trabecular meshwork maintains a balanced pressure in the anterior chamber of the eye by draining aqueous humor from the anterior chamber to Schlemm's canal located at the exterior side of the trabecular meshwork.

About two percent of people in the United States have glaucoma. Glaucoma is a group of eye diseases encompassing a broad spectrum of clinical presentations, etiologies, and treatment modalities. Glaucoma causes pathological changes in the optic nerve, visible on the optic disk, and it causes corresponding visual field loss, resulting in blindness if untreated. Lowering intraocular pressure is the major treatment goal in all glaucomas.

Glaucoma is grossly classified into two categories: closed-angle glaucoma, also known as angle closure glaucoma, and open-angle glaucoma. Open-angle glaucoma is any glaucoma in which the angle of the anterior chamber remains open, but the exit of aqueous through the trabecular meshwork is diminished. The exact cause for diminished filtration is unknown for most cases of open-angle glaucoma.

Primary open-angle glaucoma is the most common of the glaucomas, and it is often asymptomatic in the early to moderately advanced stage. Patients may suffer substantial, irreversible vision loss prior to diagnosis and treatment. However, there are secondary open-angle glaucomas which may include edema or swelling of the trabecular spaces (e.g., from corticosteroid use), abnormal pigment dispersion, or diseases such as hyperthyroidism that produce vascular congestion.

In open-angle glaucomas associated with an elevation in eye pressure (intraocular hypertension), the source of resistance to outflow mainly in the trabecular meshwork. The tissue of the trabecular meshwork allows the aqueous humor (“aqueous”) to enter Schlemm's canal, which then empties into aqueous collector channels in the posterior wall of Schlemm's canal and then into aqueous veins, which form the episcleral venous system.

Aqueous humor is a transparent liquid that fills the region between the cornea, at the front of the eye, and the lens. The aqueous humor is continuously secreted by the ciliary body around the lens, so there is a constant flow of aqueous humor from the ciliary body to the eye's front chamber. The eye's pressure is determined by a balance between the production of aqueous and its exit through the trabecular meshwork (major route) or uveal scleral outflow (minor route). The trabecular meshwork is located between the outer rim of the iris and the back of the cornea, in the anterior chamber angle. The portion of the trabecular meshwork adjacent to Schlemm's canal (the juxtacanilicular meshwork) causes most of the resistance to aqueous outflow.

Closed-angle glaucoma is caused by closure of the anterior chamber angle by contact between the iris and the inner surface of the trabecular meshwork. Closure of this anatomical angle (a phenomenon called “anatomical iris closure”) prevents normal drainage of aqueous humor from the anterior chamber of the eye. In closure-angle glaucoma, the flow-through characteristics of trabecular meshwork may be either intact or dysfunctional.

All current therapies for glaucoma are directed at decreasing intraocular pressure. Medical therapy includes topical ophthalmic drops or oral medications that reduce the production or increase the outflow of aqueous. However, these drug therapies for glaucoma are sometimes associated with significant side effects, such as headache, blurred vision, allergic reactions, death from cardiopulmonary complications, and potential interactions with other drugs.

When drug therapy fails, surgical therapy is used. Surgical therapy for open-angle glaucoma consists of laser trabeculoplasty, trabeculectomy, and implantation of aqueous shunts after failure of trabeculectomy or if trabeculectomy is unlikely to succeed. Trabeculectomy is a major surgery that is widely used and is augmented with topically applied anticancer drugs, such as 5-flurouracil or mitomycin-C to decrease scarring and increase the likelihood of surgical success. However, there is no suitable surgical therapy or device for treating closed-angle glaucoma.

Approximately 100,000 trabeculectomies are performed on Medicare-age patients per year in the United States. This number would likely increase if the morbidity associated with trabeculectomy could be decreased. The current morbidity associated with trabeculectomy consists of failure (10-15%); infection (a life long risk of 2-5%); choroidal hemorrhage, a severe internal hemorrhage from low intraocular pressure, resulting in visual loss (1%); cataract formation; and hypotony maculopathy (potentially reversible visual loss from low intraocular pressure).

For these reasons, surgeons have tried for decades to develop a workable surgery for restoring normal functions of the trabecular meshwork.

The surgical techniques that have been tried and practiced are goniotomy/trabeculotomy and other mechanical disruptions of the trabecular meshwork, such as trabeculopuncture, goniophotoablation, laser trabecular ablation, and goniocurretage. These are all major operations and are briefly described below.

Goniotomy/Trabeculotomy: Goniotomy and trabeculotomy are simple and directed techniques of microsurgical dissection with mechanical disruption of the trabecular meshwork. These initially had early favorable responses in the treatment of open-angle glaucoma. However, long-term review of surgical results showed only limited success in adults. In retrospect, these procedures probably failed due to cellular repair and fibrosis mechanisms and a process of “filling in.” Filling in is a detrimental effect of collapsing and closing in of the created opening in the trabecular meshwork. Once the created openings close, the pressure builds back up and the surgery fails.

Trabeculopuncture: Q-switched Neodymiun (Nd) YAG lasers also have been investigated as an optically invasive technique for creating full-thickness holes in trabecular meshwork. However, the relatively small hole created by this trabeculopuncture technique exhibits a filling-in effect and fails.

Goniophotoablation/Laser Trabecular Ablation: Goniophotoablation is disclosed by Berlin in U.S. Pat. No. 4,846,172 and involves the use of an excimer laser to treat glaucoma by ablating the trabecular meshwork. This was demonstrated not to succeed by clinical trial. Hill et al. used an Erbium:YAG laser to create full-thickness holes through trabecular meshwork (Hill et al., Lasers in Surgery and Medicine 11:341-346, 1991). This technique was investigated in a primate model and a limited human clinical trial at the University of California, Irvine. Although morbidity was zero in both trials, success rates did not warrant further human trials. Failure was again from filling in of surgically created defects in the trabecular meshwork by repair mechanisms. Neither of these is a viable surgical technique for the treatment of glaucoma.

Goniocurretage: This is an ab interno (from the inside), mechanically disruptive technique that uses an instrument similar to a cyclodialysis spatula with a microcurrette at the tip. Initial results were similar to trabeculotomy: it failed due to repair mechanisms and a process of filling in.

Although trabeculectomy is the most commonly performed filtering surgery, viscocanulostomy (VC) and non-penetrating trabeculectomy (NPT) are two new variations of filtering surgery. These are ab externo (from the outside), major ocular procedures in which Schlemm's canal is surgically exposed by making a large and very deep scleral flap. In the VC procedure, Schlemm's canal is cannulated and viscoelastic substance injected (which dilates Schlemm's canal and the aqueous collector channels). In the NPT procedure, the inner wall of Schlemm's canal is stripped off after surgically exposing the canal.

Trabeculectomy, VC, and NPT involve the formation of an opening or hole under the conjunctiva and scleral flap into the anterior chamber, such that aqueous humor is drained onto the surface of the eye or into the tissues located within the lateral wall of the eye. These surgical operations are major procedures with significant ocular morbidity. When trabeculectomy, VC, and NPT are thought to have a low chance for success, a number of implantable drainage devices have been used to ensure that the desired filtration and outflow of aqueous humor through the surgical opening will continue. The risk of placing a glaucoma drainage device also includes hemorrhage, infection, and diplopia (double vision).

Examples of implantable shunts and surgical methods for maintaining an opening for the release of aqueous humor from the anterior chamber of the eye to the sclera or space beneath the conjunctiva have been disclosed in, for example, U.S. Pat. No. 6,059,772 to Hsia et al. and U.S. Pat. No. 6,050,970 to Baerveldt.

All of the above embodiments and variations thereof have numerous disadvantages and moderate success rates. They involve substantial trauma to the eye and require great surgical skill in creating a hole through the full thickness of the sclera into the subconjunctival space. The procedures are generally performed in an operating room and have a prolonged recovery time for vision.

The complications of existing filtration surgery have inspired ophthalmic surgeons to find other approaches to lowering intraocular pressure.

The trabecular meshwork and juxtacanilicular tissue together provide the majority of resistance to the outflow of aqueous and, as such, are logical targets for surgical removal in the treatment of open-angle glaucoma. In addition, minimal amounts of tissue are altered and existing physiologic outflow pathways are utilized. Co-pending U.S. patent application Ser. No. 09/549,350, filed Apr. 14, 2000, and entitled “APPARATUS AND METHOD FOR TREATING GLAUCOMA,” discloses ab interno surgical procedures and their associated devices, the entire contents of which are hereby incorporated by reference herein.

On the other hand, in angle closure glaucoma, the flow pathway between the anterior chamber and trabecular meshwork provides the majority of resistance to the outflow of aqueous, and as such, is a logical target for placing a hollow stenting glaucoma device for aqueous outflow to enter trabecular meshwork and thereafter enter Schlemm's canal, which then empties into aqueous collector channels in the posterior wall of Schlemm's canal and then into aqueous veins, which form the episcleral venous system.

Glaucoma reportedly remains a leading cause of blindness (Arch. Ophthalm. pp. 118:412, 2000), and filtration surgery remains an effective, important option in controlling the disease. However, modifying existing filtering surgery techniques in any profound way to increase their effectiveness appears to have reached a dead end. The article further states that the time has come to boldly examine new surgical approaches that may provide better and safer care for patients with glaucoma.

Therefore, there is a great clinical need for the treatment of angle closure glaucoma by a method that is faster, safer, and less expensive than currently available modalities.

### SUMMARY OF THE INVENTION

Glaucoma surgical morbidity would greatly decrease if one were to bypass the focal resistance to outflow of aqueous only at the point of resistance, and to utilize remaining, healthy aqueous outflow mechanisms. This is in part because episcleral aqueous humor exerts a backpressure that prevents intraocular pressure from going too low, and one could thereby avoid hypotony. Thus, such a surgical operation would virtually eliminate the risk of hypotony-related maculopathy and choroidal hemorrhage. Furthermore, visual recovery would be very rapid, and the risk of infection would be very small (a reduction from 2-5% to about 0.05%).

One technique performed in accordance with the invention may be referred to generally as “trabecular bypass surgery.” Advantages of the invention include lowering intraocular pressure in a manner which is simple, effective, disease site-specific, and can potentially be performed on an outpatient basis.

In accordance with one embodiment, a glaucoma treatment device is provided for directing the flow of aqueous humor and reducing intraocular pressure for angle closure glaucoma. The glaucoma device comprises an aqueous transporting element for transporting aqueous humor to bypass dysfunctional anatomical iris closure and restoring existing outflow pathways of the anatomical iris closure. The aqueous transporting element has an inlet end and an outlet end, wherein the inlet end is positioned inside an anterior chamber of an eye beyond an edge of the dysfunctional anatomic iris closure and the outlet end is positioned in proximity of trabecular meshwork of the eye. The device also serves to stent the space between the iris and the inner surface of the cornea.

In accordance with one aspect of the invention, trabecular bypass surgery (TBS) creates an opening, a slit, or a hole through trabecular meshwork with minor microsurgery. TBS has the advantage of a much lower risk of choroidal hemorrhage and infection than prior techniques, and it uses existing physiologic outflow mechanisms. In some aspects, this surgery can potentially be performed under topical or local anesthesia on an outpatient basis with rapid visual recovery. To prevent “filling in” of the hole, a biocompatible glaucoma device may be placed within the hole, serving as a stenting glaucoma device. The hole on trabecular meshwork may also serve as an anchoring spot for the stenting glaucoma device.

In some embodiments, the device may be positioned across trabecular meshwork alone, without extending into the eye wall or sclera. For angle closure glaucoma, the inlet end of the device is exposed to the anterior chamber of the eye while the outlet end is positioned either at the inner surface or at the exterior surface of the trabecular meshwork.

In another embodiment, the outlet end is positioned at the exterior surface of the trabecular meshwork and into the fluid collection channels of the existing outflow pathways. In still another embodiment, the outlet end is positioned in Schlemm's canal. In yet another embodiment, the outlet end enters into fluid collection channels (e.g., aqueous collector channels) up to the level of the aqueous veins, with the device inserted in a retrograde or antegrade fashion.

In some embodiments, the device is made of biocompatible material, which is hollow and/or has at least one exterior trough, to allow the flow of aqueous humor. In other embodiments, the device is made of biocompatible porous material that imbibes aqueous humor. One or more materials for the device may be selected from the following material types: porous material, semi-rigid material, soft material, hydrophilic material, hydrophobic material, hydrogel, elastic material, biodegradable material, bioresorbable material, and the like.

One or more materials for the glaucoma device may be selected from the following: polyvinyl alcohol, polyvinyl pyrolidone, collagen, heparinized collagen, chemically treated collagen, polytetrafluoroethylene, expanded polytetrafluoroethylene, fluorinated polymer, fluorinated elastomer, flexible fused silica, silicone, polyurethane, poly(methyl methacrylate), acrylic, polyolefin, polyester, polysilicon, polypropylene, hydroxyapetite, titanium, gold, silver, platinum, biodegradable material, bioresorable material, and mixture thereof. Other suitable types and materials for the device may be used in accordance with the invention and will be apparent to those of skill in the art.

In accordance with a further aspect of the invention, a portion of the device is relatively soft and somewhat curved at its outlet section to fit into the existing outflow pathways, such as Schlemm's canal. The outlet section may be curved around a curve center, and the middle section may extend substantially along a plane that contains the curve center. All or a portion of the cross section of one or more lumens may be in an elliptical (e.g., oval) shape. Furthermore, the outlet section inside the outflow pathway may have an appropriate shape, e.g., with a protuberance or barb projecting from it, to stabilize the device in place without undue suturing.

One aspect of the invention includes a method of placing a glaucoma device into an opening through trabecular meshwork and into an outflow pathway for aqueous humor. This glaucoma device includes an inlet section, an outlet section, and a middle section between the inlet section and the outlet section. The glaucoma device also includes at least one lumen that extends within at least one of the three sections for transmitting aqueous humor, and the outlet section is substantially perpendicular to the middle section. The outlet section includes a first outlet end and a second outlet end. In this aspect of the invention, the method includes advancing the first outlet end of the outlet section through the opening into a first part of the outflow pathway, and advancing the second outlet end of the outlet section through the opening into a second part of the outflow pathway.

Another aspect of the invention includes a method of placing a hollow stenting glaucoma device between the iris and the inner surface of the cornea for aqueous to flow from anterior chamber to the proximity of trabecular meshwork. The stenting glaucoma device is either stabilized within the sandwich of the iris and the cornea, or stabilized by placing a portion of the stenting glaucoma device inside the opening of trabecular meshwork or even into Schlemm's canal.

Among the advantages of trabecular bypass surgery in accordance with the invention is its simplicity. The microsurgery may potentially be performed on an outpatient basis with rapid visual recovery and greatly decreased morbidity. There is a lower risk of infection and choroidal hemorrhage, and there is a faster recovery, than with previous techniques.

For purposes of summarizing the invention, certain aspects, advantages and novel features of the invention have been described herein above. Of course, it is to be understood that not necessarily all such advantages may be achieved in accordance with any particular embodiment of the invention. Thus, the invention may be embodied or carried out in a manner that achieves or optimizes one advantage or group of advantages as taught or suggested herein without necessarily achieving other advantages as may be taught or suggested herein.

All of these embodiments are intended to be within the scope of the invention herein disclosed. These and other embodiments of the invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

Having thus summarized the general nature of the invention and some of its features and advantages, certain preferred embodiments and modifications thereof will become apparent to those skilled in the art from the detailed description herein having reference to the figures that follow, of which:

FIG. 1 is a sagittal sectional view of an eye;

FIG. 2 is an enlarged cross-sectional partial view of an anterior chamber of the eye of FIG. 1;

FIG. 3 is an oblique elevational view of a glaucoma device having features and advantages in accordance with one embodiment of the invention;

FIG. 4 is a front end view, along line 4-4, of an elongate tubular section of the glaucoma device of FIG. 3;

FIG. 5 is a perspective partial view of an anterior chamber of an eye illustrating the positioning of the glaucoma device of FIG. 3 therein in accordance with one embodiment of the invention;

FIG. 6 is an illustration of a method of placement of the glaucoma device of FIG. 3 in an eye in accordance with one embodiment of the invention;

FIG. 7 is an oblique elevational view of a glaucoma device having features and advantages in accordance with another embodiment of the invention; and

FIG. 8 is a perspective partial view of an anterior chamber of an eye illustrating the positioning of the glaucoma device of FIG. 7 therein in accordance with one embodiment of the invention.

### DETAILED DESCRIPTION OF THE PREFERRED EMBODIMENTS

The preferred embodiments of the invention described herein relate particularly to surgical and therapeutic treatment of glaucoma through reduction of intraocular pressure. More particularly, these embodiments relate to an apparatus and methods thereof for the treatment of angle closure glaucoma by microsurgery.

While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described herein.

Angle closure glaucoma is partly caused by closure of the anterior chamber angle by contact between the iris and the inner surface of the trabecular meshwork. Closure of this anatomical angle (also referred to as “dysfunctional anatomical iris closure” herein) prevents normal drainage of aqueous humor from the anterior chamber of the eye.

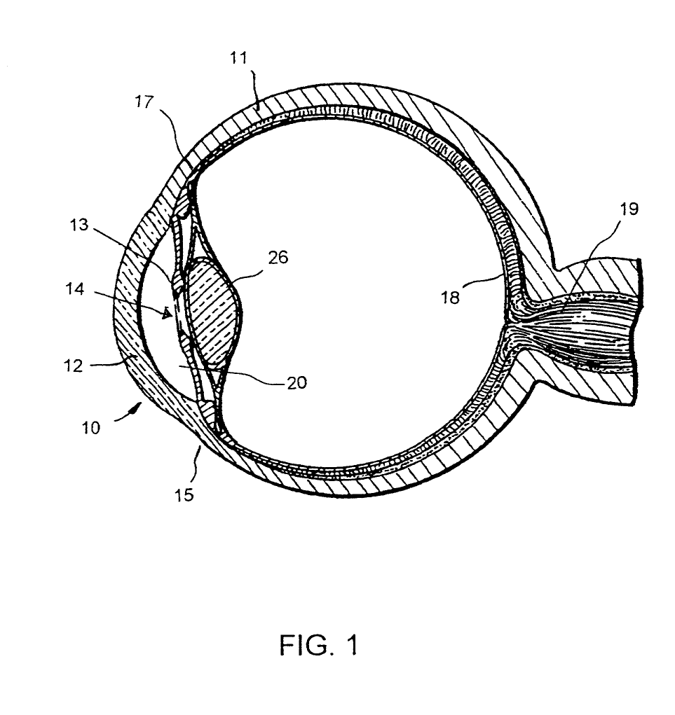

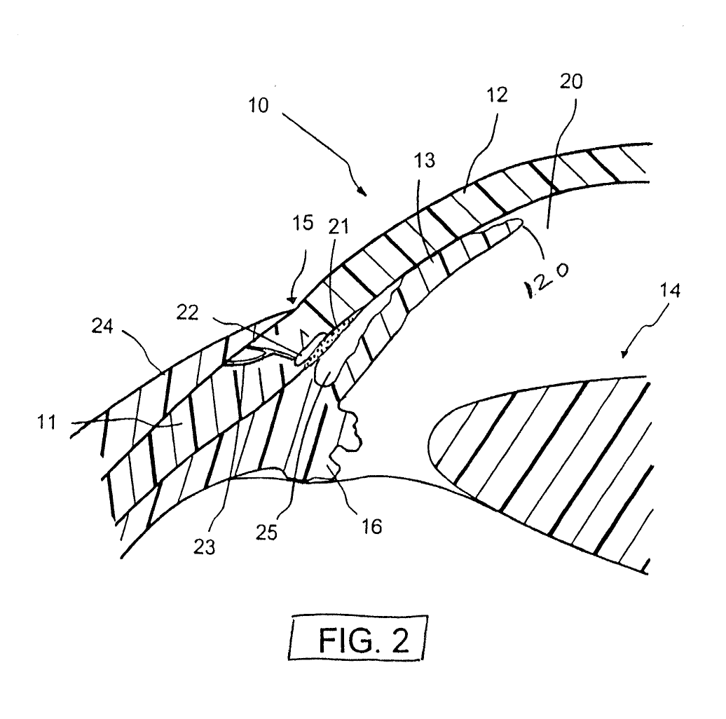

FIG. 1 is a sagittal sectional view of an eye 10, while FIG. 2 is a close-up view showing the relative anatomical locations of a trabecular meshwork 21, an anterior chamber 20, and Schlemm's canal 22. A sclera 11 is a thick collagenous tissue which covers the entire eye 10 except that portion which is covered by a cornea 12.

Referring to FIGS. 1 and 2, the cornea 12 is a thin transparent tissue that focuses and transmits light into the eye and through a pupil 14, which is a generally circular hole in the center of an iris 13 (colored portion of the eye). The cornea 12 merges into the sclera 11 at a juncture referred to as a limbus 15. A ciliary body 16 extends along the interior of the sclera 11 and is coextensive with a choroid 17. The choroid 17 is a vascular layer of the eye 10, located between the sclera 11 and a retina 18. An optic nerve 19 transmits visual information to the brain and is the anatomic structure that is progressively destroyed by glaucoma.

Still referring to FIGS. 1 and 2, the anterior chamber 20 of the eye 10, which is bound anteriorly by the cornea 12 and posteriorly by the iris 13 and a lens 26, is filled with aqueous humor (also referred to as “aqueous” herein). Aqueous is produced primarily by the ciliary body 16, then moves anteriorly through the pupil 14 and reaches an anterior chamber angle 25, formed between the iris 13 and the cornea 12.

As best illustrated by the drawing of FIG. 2, in a normal eye, the aqueous is removed from the anterior chamber 20 through the trabecular meshwork 21. Aqueous passes through the trabecular meshwork 21 into Schlemm's canal 22 and thereafter through a plurality of aqueous veins 23, which merge with blood-carrying veins, and into systemic venous circulation. Intraocular pressure is maintained by an intricate balance between secretion and outflow of aqueous in the manner described above. Glaucoma is, in most cases, characterized by an excessive buildup of aqueous humor in the anterior chamber 20 which leads to an increase in intraocular pressure. Fluids are relatively incompressible, and thus intraocular pressure is distributed relatively uniformly throughout the eye 10.

As shown in FIG. 2, the trabecular meshwork 21 is adjacent a small portion of the sclera 11. Exterior to the sclera 11 is a conjunctiva 24. Traditional procedures that create a hole or opening for implanting a device through the tissues of the conjunctiva 24 and sclera 11 involve extensive surgery, as compared to surgery for implanting a device which ultimately resides entirely within the confines of the sclera 11 and cornea 12, as is performed in accordance with one aspect of the invention. As discussed further below, glaucoma devices for establishing an outflow pathway, in accordance with some preferred embodiments, are positioned in proximity of the trabecular meshwork 21 and in-between the iris 13 and cornea 12.

For angle closure glaucoma, an elongate device (either an elongate tubular member type or an annular member type) for transmitting aqueous from the anterior chamber to the trabecular meshwork to bypass the analytical iris closure may be implanted in accordance with one embodiment of the invention. The elongate device serves as a hollow stenting glaucoma device to be placed at dysfunctional anatomical iris closure for restoring existing outflow pathways of the anatomical iris closure.

Tubular Glaucoma Device Featuring an Open Trough Configuration

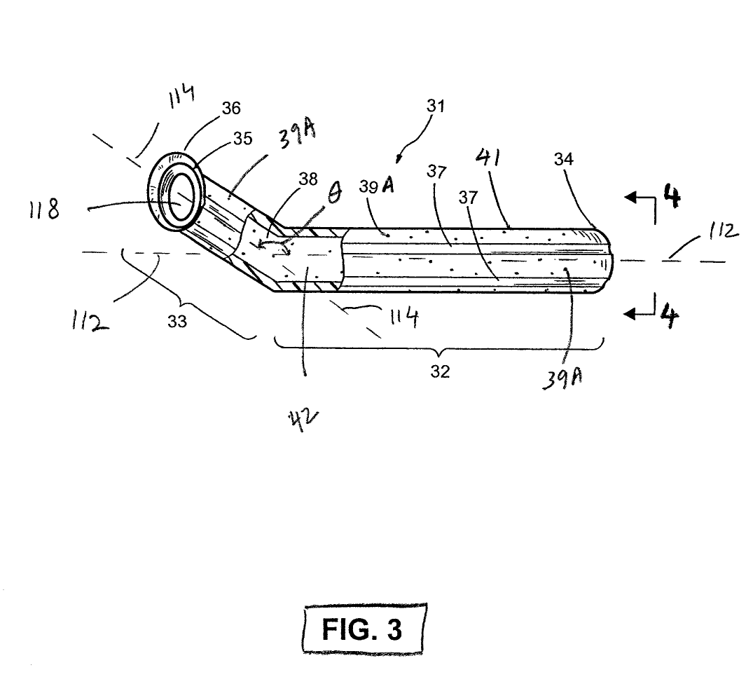

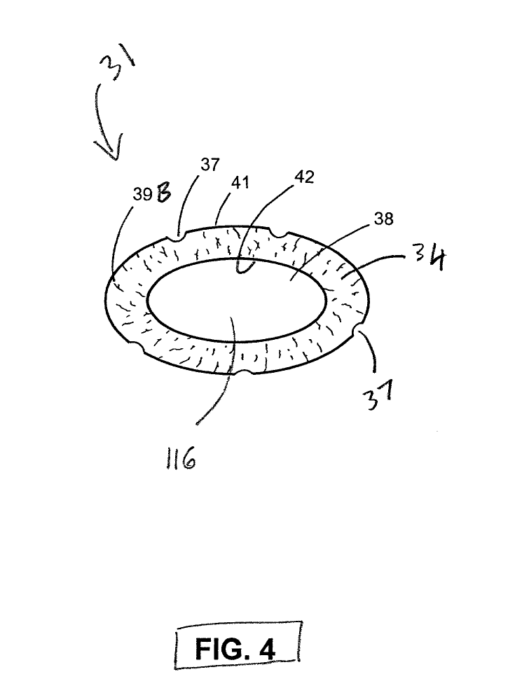

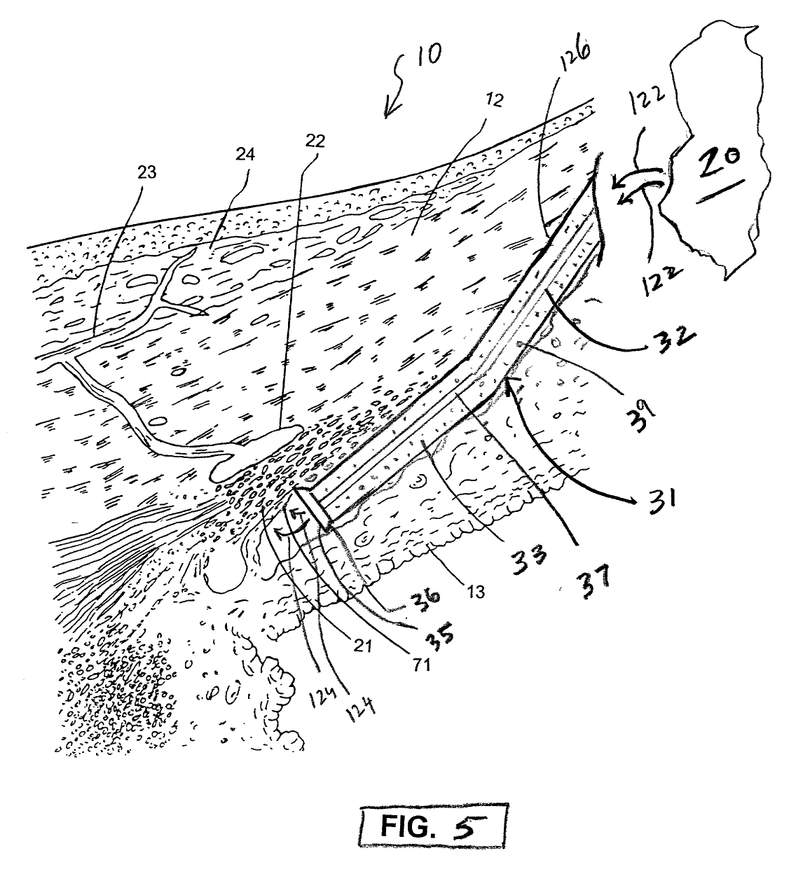

FIGS. 3 and 4 show different views of a glaucoma device 31 comprising a generally elongate tubular member and having features and advantages in accordance with one embodiment. FIG. 5 is a perspective partial view of an anterior chamber 20 of an eye 10 illustrating the positioning of the glaucoma device 31 therein in accordance with one embodiment.

Referring in particular to the illustrated embodiment of FIG. 3, the elongate tubular member device 31 comprises an elongate tubular section 32 having a generally longitudinal axis 112 and an optional inserting section 33 having a generally longitudinal axis 114. As discussed in further detail below, the sections 32, 33 (and/or axes 112, 114) are angled relative to one another by a predetermined angle.

Referring to FIGS. 3-5, in one embodiment, the glaucoma device 31 comprises an integral unit. In another embodiment, the glaucoma device 31 is formed by mechanically connecting two or more of its components to one another, for example, by mechanically connecting the elongate tubular section 32 and the inserting section 33. As the skilled artisan will appreciate any one of a number of techniques may be used to connect the components of the device 31. These may include, without limitation, welding, gluing and the like.

In the illustrated embodiment of FIGS. 3-5, and as best seen in FIG. 4, the glaucoma device 31 has a generally elliptical or oval shape or cross-section. In another embodiment, one or more selected portions of the glaucoma device 31 may have a generally elliptical or oval shape or cross-section. In other embodiments, selected portions of the glaucoma device 31 may efficaciously be shaped in modified manners, as required or desired, giving due consideration to the goals of achieving one or more of the benefits as disclosed, taught or suggested herein. For example, selected portions of the glaucoma device 31 may have a circular shape or cross-section among other suitable polygonal or non-polygonal shapes or cross-sections and combinations thereof.

In the illustrated embodiment of FIGS. 3-5, the glaucoma device 31 has a proximal or inlet end 34, a distal or outlet end 35 and an outer surface 41. The glaucoma device 31 comprises a lumen 38 extending therethrough for transport of aqueous and which has an inner luminal surface 42, an inlet opening or orifice 116 at the device proximal end 34 and an outlet opening or orifice 118 at the device distal end 35.

Preferably, the device 31 itself comprises a porous material. In the illustrated embodiment of FIGS. 3-5, and as shown in FIG. 3, the outer surface 41 of the elongate tubular section 32 and/or the inserting section 33 may have a plurality of tiny holes or pores 39A for aqueous to diffuse into and out of the device to facilitate efficient transportation of aqueous humor. The holes or pores 39A provide fluid communication between the aqueous at the outer surface 41 and the device lumen 38 and extend form the outer surface 41 to the inner luminal surface 42 generally towards the direction of the axis 112 and/or axis 114.

Referring in particular to FIG. 3, in one embodiment, the pores 39A extend generally radially towards the axis 112 and/or the axis 114. One or more of the pores 39A may also interconnect with one or more other pores 39A, as needed or desired.

Preferably, the device 31 comprises a plurality of tiny holes or pores at the proximal end 34 and the distal end 35. In the illustrated embodiment of FIGS. 3-5, and as shown in FIG. 4 for the proximal end 34, these pores 39B are located between the outer surface 41 and the luminal surface 42 for aqueous transfusion.

Referring in particular to FIGS. 3 and 4, in one embodiment, the pores 39B facilitate aqueous transportation through the device 31 in a direction generally parallel to the axis 112 and/or the axis 114. One or more of the pores 39B may also interconnect with one or more other pores 39B, as needed or desired. One or more of the pores 39B may also be in fluid communication with the lumen 38, as needed or desired. One or more of the pores 39B may also be in fluid communication with or interconnect with one or more of the pores 39A. Advantageously, the positioning and/or interaction between the lumen 38, pores 39A and/or the pores 39B creates a suitable network of fluid passageways within the body of the device 31 which facilitates efficient transport and/or transfusion of aqueous humor.

As best seen in FIG. 3, a trumpet-type flange 36 is optionally provided at the distal end 35. Advantageously, the flange 36 promotes outflow characteristics and facilitates in the efficient transport of aqueous through the device 31. In one embodiment, the flange 36 is integrally formed into the device 31. In another embodiment, the flange 36 is mechanically connected or attached to the distal end 35. As the skilled artisan will appreciate any one of a number of techniques may be used to connect the flange to the device distal end 35. These may include, without limitation, welding, gluing and the like.

In the illustrated embodiment of FIGS. 3-5, the outer surface 41 of the device 31 comprises a plurality of generally longitudinal troughs 37. The open troughs 37 and the lumen 38 of the device 31 generally provide main passageways for aqueous transmission.

In the illustrated embodiment of FIGS. 3-5, and as best seen in FIG. 4, the troughs 37 are generally C-shaped or semi-circular. In other embodiments, one or more of the troughs 37 and/or selected portions thereof may be efficaciously shaped in modified manners, as required or desired, giving due consideration to the goals of providing efficient aqueous transmission and/or of achieving one or more of the benefits as disclosed, taught or suggested herein. For example, one or more of the troughs 37 and/or selected portions thereof may be generally U-shaped, V-Shaped, rectangular, semi-elliptical among other suitable polygonal or non-polygonal shapes and combinations thereof.

In the illustrated embodiment of FIGS. 3-5, and as shown in FIG. 4, the device 31 comprises five troughs 37. In another embodiment, the device 31 comprises between two and ten troughs 37. In yet another embodiment, the device 31 comprises between one and twenty troughs 37. In other embodiments, the device 31 may efficaciously comprise fewer or more troughs 37, as required or desired, giving due consideration to the goals of providing efficient aqueous transmission and/or of achieving one or more of the benefits as disclosed, taught or suggested herein.

The open troughs 37 (FIGS. 3-5) of the device 31 may be efficaciously arranged on the outer surface 41 in a variety of manners, as required or desired, giving due consideration to the goals of providing efficient aqueous transmission and/or of achieving one or more of the benefits as disclosed, taught or suggested herein. For example, the troughs 37 may be arranged in a generally symmetrical or asymmetrical fashion and/or substantially equidistantly from adjacent troughs 37. In a modified embodiment, one or more of the troughs 37 may interconnect with one or more of the other troughs 37.

In the illustrated embodiment of FIGS. 3-5, and as best seen in FIGS. 3 and 4, the lumen 38 has a generally elliptical or oval shape or cross-section. In another embodiment, one or more selected portions of the lumen 31 may have a generally elliptical or oval shape or cross-section. In a further embodiment, the device 31 may comprise more than one or a plurality of lumens, as required or desired, giving due consideration to the goals of providing efficient aqueous transport and/or of achieving one or more of the benefits as disclosed, taught or suggested herein.

In other embodiments, selected portions of one or more of the lumens 38 may efficaciously be shaped in modified manners, as required or desired, giving due consideration to the goals of providing efficient aqueous transport and/or of achieving one or more of the benefits as disclosed, taught or suggested herein. For example, selected portions of one or more of the lumens 38 may have a circular shape or cross-section among other suitable polygonal or non-polygonal shapes or cross-sections and combinations thereof.

The glaucoma device 31 (FIGS. 3-5) may be made, manufactured or fabricated by a wide variety of techniques. These include, without limitation, molding, thermo-forming, or other micro-machining techniques, among other suitable techniques.

Referring in particular to FIGS. 3-5, the glaucoma device 31 is preferably biocompatible so that any inflammation caused by irritation between the outer surface of the device 31 and surrounding tissue is minimal. The device 31 may comprise a biocompatible material, such as medical grade silicone, e.g., Silastic™, available from Dow Corning Corporation of Midland, Mich.; or polyurethane, e.g., Pellethane™, also available from Dow Corning Corporation.

Biocompatible material (biomaterial) suitable for the manufacturing the device 31 may include polyvinyl alcohol, polyvinyl pyrolidone, collagen, heparinized collagen, chemically treated collagen, polytetrafluoroethylene, expanded polytetrafluoroethylene, fluorinated polymer, fluorinated elastomer, flexible fused silica, silicone, polyurethane, poly(methyl methacrylate), acrylic, polyolefin, polyester, polysilicon, polypropylene, hydroxyapetite, titanium, gold, silver, platinum, biodegradable material, bioresorable material, a mixture of two or more of the above biocompatible materials or a mixture of other biocompatible materials, and the like.

In a further embodiment, a composite biocompatible material may be used, wherein a surface material may be used in addition to one or more of the aforementioned materials. Such a surface material may include polytetrafluoroethylene (“PTFE”) (such as Teflon™), polyimide, hydrogel, heparin, hydrophilic coating, therapeutic drugs (such as beta-adrenergic antagonists, other anti-glaucoma drugs, or antibiotics), a combination thereof, and the like.

The glaucoma device of FIGS. 3-5 may be efficaciously dimensioned and sized in a variety of manners. The length of the device 31 typically depends on the distance between the anterior chamber 20 and outflow passageways (e.g., trabecular meshwork 21 or a vein) into which the device 31 drains aqueous humor. When the device 31 is placed within the eye 10, the proximal or inlet end 34 of the elongate tubular section 32 is preferably beyond or close to the edge 120 (see FIG. 2) of the iris 13 whereas aqueous is in communication from the anterior chamber 20 into the device 31, as indicated generally by the arrows 122 (FIG. 5). Since in most cases, the trabecular meshwork 21 for angle closure glaucoma is still functional or intact to certain degree, the distal or outlet end 35 of the inserting section 33 may be located at about the inner surface 71 (as shown in FIG. 5) of trabecular meshwork 21 for aqueous transportation using existing outflow pathways, as indicated generally by the arrows 124 (FIG. 5).

In one embodiment, the device 31 has a length of about 5 millimeters (mm). In another embodiment, the device 31 has a length in the range from about 2.5 mm to about 7.5 mm. In yet another embodiment, the device 31 has a length in the range from about 0.5 mm to about 10 mm. Other suitable lengths may also be utilized with efficacy, as needed or desired.

The device 31 and/or the tubular section 32 also serves to stent the space between the iris 13 and the inner surface 126 (FIG. 5) of the cornea 12. In one embodiment, the device 31 has a diameter or major diameter of about 250 microns (μm). In another embodiment, the device 31 has a diameter or major diameter in the range from about of about 200 μm to about 300 μm. In yet another embodiment, the device 31 has a diameter or major diameter in the range from about 100 μm to about 400 μm. In still another embodiment, the device 31 has a diameter or major diameter in the range from about 30 μm to about 500 μm. The device 31 preferably has a minor diameter in the range from about 25% of the device major diameter to about the same as or about 100% of the major diameter (that is, a circular cross-section). Other suitable device diameters may also be utilized with efficacy, as needed or desired.

In one embodiment, the device lumen 38 has a diameter or major diameter of about 100 microns (μm). In another embodiment, the lumen 38 has a diameter or major diameter in the range from about 50 μm to about 200 μm. In yet another embodiment, the lumen 38 has a diameter or major diameter in the range from about 20 μm to about 250 μm. The device lumen 38 preferably has a minor diameter in the range from about 25% of the lumen major diameter to about the same as or about 100% of the major diameter (that is, a circular cross-section). Other suitable lumen diameters may also be utilized with efficacy, as needed or desired.

Referring in particular to FIG. 3, the angle between the longitudinal axis 112 of the elongate tubular section 32 and the longitudinal axis 114 of the inserting section 33 is denoted by θ. In some embodiments, the angle θ is appropriately selected so that the inserting section 33 may be optionally inserted into a cut slit of the trabecular meshwork 21 while the elongate tubular section 32 lies between the iris 13 and the inner surface 126 (as shown in FIG. 5) of the cornea 12. Furthermore, the outlet or inserting section 33, particularly in the embodiments when it is placed inside the outflow pathway, may have an appropriate shape, e.g., with a protuberance, barb, deeply threaded shank or the like projecting from it, to stabilize the device 31 in place without undue suturing.

In one embodiment, θ is about 175° (degrees). In another embodiment, θ is about 180° (that is, the elongate tubular section 32 and the inserting section 33 are generally coaxially aligned). In yet another embodiment, θ is in the range from about 150° to about 180°. In still another embodiment, θ is in the range from about 120° to about 185°. Other suitable values for θ may also be utilized with efficacy, as needed or desired.

As indicated above, in some embodiments, an opening or perforation in the trabecular meshwork 21 is created for anchoring the inserting section 33 inside the trabecular meshwork 21. This opening can be created by laser, a knife, or other surgical cutting instrument. The opening may advantageously be substantially horizontal, i.e., extending longitudinally in the same direction as the circumference of the limbus 15. Other opening directions may also be efficaciously used, such as horizontal or at any angle that is appropriate for inserting the glaucoma device 31 through the trabecular meshwork 21 and into Schlemm's canal or another outflow pathway, as will be apparent to those of skill in the art.

In one embodiment, the method of forming an opening in the trabecular meshwork 21 may comprise making an incision with a microknife, a pointed guidewire, a sharpened applicator, a screw-shaped applicator, an irrigating applicator, or a barbed applicator. Alternatively, or in addition, the trabecular meshwork 21 may be dissected with an instrument similar to a retinal pick, or a microcurrette. In another embodiment, the opening may be created by fiberoptic laser ablation. In one preferred embodiment, a device delivery applicator comprising an opening-creating capability is used to facilitate creating an opening in the trabecular meshwork 21 and inserting the glaucoma device 31 in one operating procedure.

A further aspect of the invention includes methods for increasing aqueous humor outflow in an eye 10 of a patient to reduce intraocular pressure therein. One method involves placing the glaucoma device 31 into the anterior chamber 20 of the eye 10 for reducing intraocular pressure in a patient having a dysfunctional anatomical iris closure in angle closure glaucoma. The method generally comprises advancing the glaucoma device 31 using a delivery applicator through an incision of the eye 10 and positioning the device 31 at about the dysfunctional anatomical iris closure. Then aqueous humor is transmitted through the device 31 and enters the trabecular meshwork 21, from the deep side to the superficial side of the trabecular meshwork 21. This “transmitting” of aqueous humor is, in one aspect of the invention, preferably passive, i.e., aqueous humor is allowed to flow out of the anterior chamber 20 due to the pressure gradient between the anterior chamber 20 and the aqueous venous system including the aqueous veins 23.

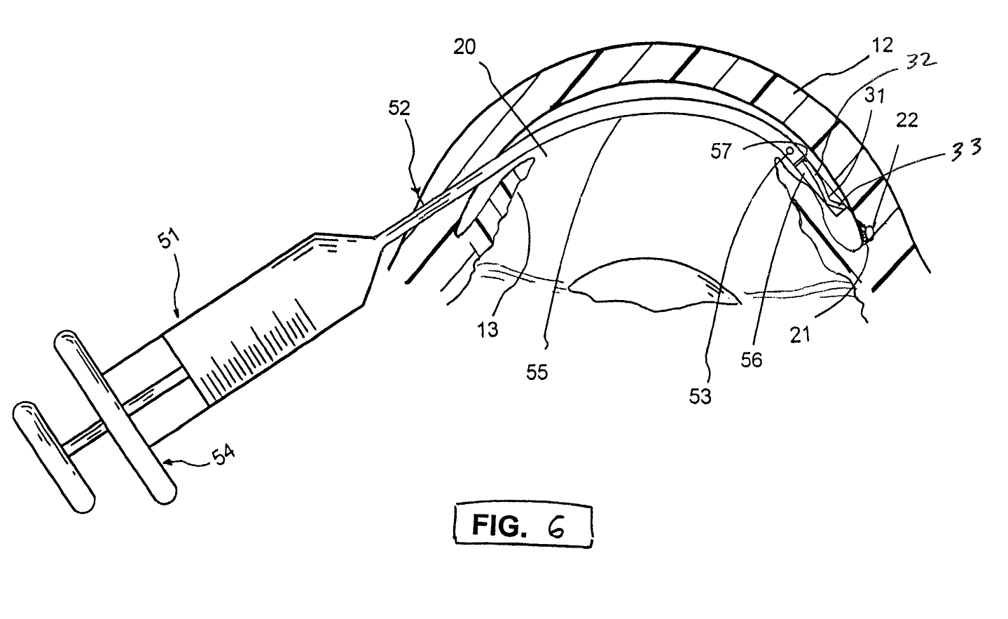

FIG. 6 shows an aspect of placing the glaucoma device 31 at the implantation site. An irrigating knife or device delivery applicator 51 is provided, which, in some embodiments, comprises a syringe portion 54 and a cannula portion 55. The cannula portion 55 may be curved to facilitate inserting the device 31 into the anatomical iris closure. The distal section of the cannula portion 55 has at least one optional irrigating hole 53 and a distal space 56 for holding the device 31. The proximal end 57 of the lumen of the distal space 56 is, in one embodiment, sealed off from, and thus substantially not in communication with, the remaining lumen of the cannula portion 55. In this embodiment, the device 31 is placed on the delivery applicator 51 and advanced to the implant site, wherein the delivery applicator 51 holds the device 31 securely during delivery and releases it when the surgeon chooses to deploy the device 31. An optional cutting knife at the distal end of the applicator 51 renders the two steps of slitting and device deployment in one operating procedure.

In some embodiments of trabecular meshwork surgery in accordance with the invention, the patient is placed in the supine position, prepped, draped, and anesthetized as necessary. In one embodiment, a small (typically less than about 1 mm) incision 52 (see FIG. 6), which may be self-sealing, is made through the cornea 12. Through this incision, the trabecular meshwork 21 is accessed, and an incision is made in the trabecular meshwork 21 with an irrigating knife. The device 31 is then advanced through the corneal incision 52 across the anterior chamber 20, while the device 31 is held in an irrigating applicator 51, under gonioscopic, microscopic, or endoscopic guidance. After the device 31 is implanted in place, the applicator 51 is withdrawn and the surgery concluded. The irrigating knife may be within a size range of about 16 to about 40 gauge, and, in some embodiments, preferably about 30 gauge.

It is one preferred embodiment that the elongate tubular section 33 is placed, anchored, or implanted inside the anterior chamber 20 so that adequate aqueous humor is transported from the anterior chamber 20 through tissue of the trabecular meshwork 21 to enter Schlemm's canal 22, which then empties into aqueous collector channels in the posterior wall of Schlemm's canal 22 and then into aqueous veins 23 (see FIG. 2), which form the episcleral venous system.

Annular Glaucoma Device Featuring an Open Trough Configuration

In some preferred embodiments, and as indicated above, the glaucoma device is an annular member that is selected from a group comprising an annual, a semi-annular, a ring, an oval ring, or a semi-open ring device, to transport aqueous humor from the anterior chamber to about proximity of the trabecular meshwork. This device also serves to stent the space between the iris and the inner surface of the cornea.

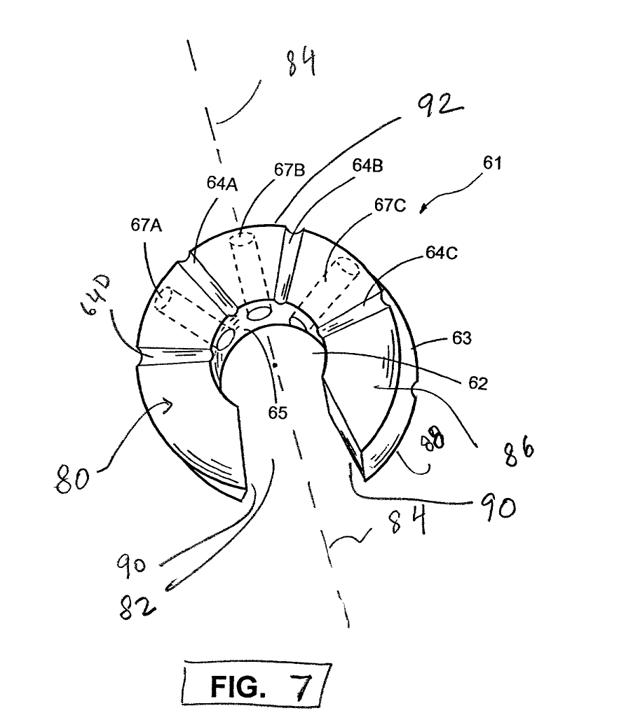

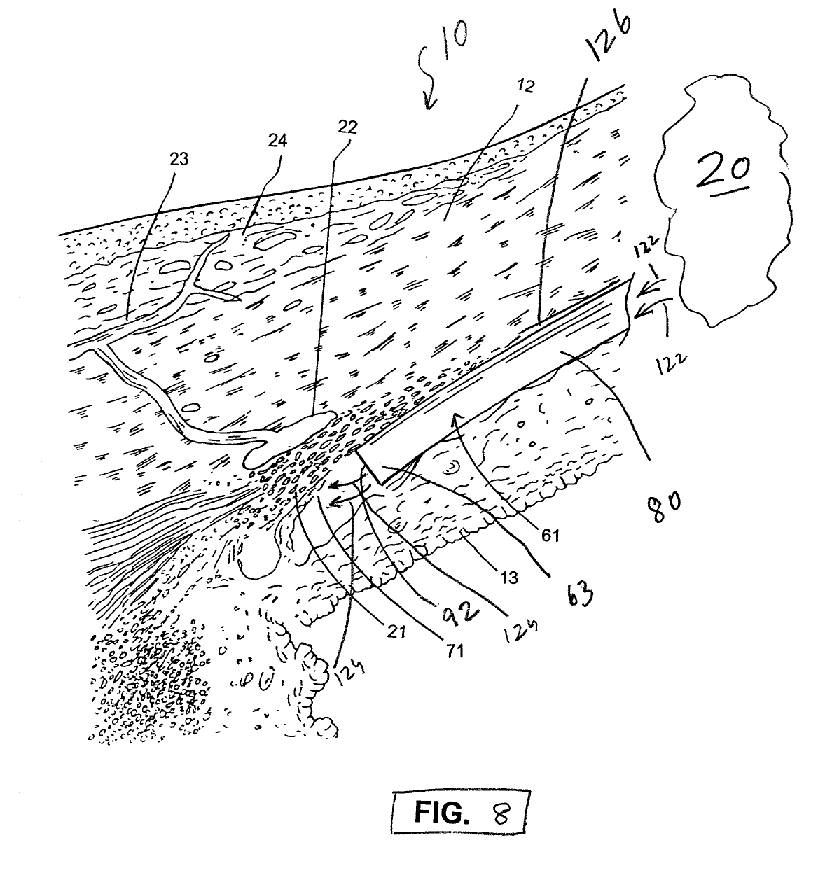

FIG. 7 is an oblique elevational view of a glaucoma device 61 comprising a generally elongate annular member and having features and advantages in accordance with one embodiment. FIG. 8 is a perspective partial view of an anterior chamber 20 of an eye 10 illustrating the positioning of the glaucoma device 61 therein in accordance with one embodiment.

In the illustrated embodiment of FIGS. 7 and 8, the annular member device 61 comprises a semi-annular ring-like main body portion 80 having a cut-off portion 82, a generally central inner space, cavity or passage 62 and a generally central axis 84. Optionally, in some embodiments the device 61 may comprise an inserting section as discussed above in connection with, for example, FIG. 3, for insertion into a cut slit of the trabecular meshwork 21 while the main body portion 80 lies between the iris 13 and the inner surface 126 (as shown in FIG. 7) of the cornea 12. Furthermore, the outlet or inserting section, particularly in the embodiments when it is placed inside the outflow pathway, may have an appropriate shape, e.g., with a protuberance, barb, deeply threaded shank or the like projecting from it, to stabilize the device 61 in place without undue suturing.

Referring to FIGS. 7 and 8, in one embodiment, the glaucoma device 61 comprises an integral unit. In another embodiment, the glaucoma device 61 is formed by mechanically connecting two or more of individual components to one another, for example, by mechanically connecting the main body portion 80 and the optional inserting section. As the skilled artisan will appreciate any one of a number of techniques may be used to connect the components of the device 61. These may include, without limitation, welding, gluing and the like.

Referring in particular to FIG. 7, the stenting glaucoma device 61 generally comprises an inner or interior surface 65, an outer or exterior surface 63, an upper surface 86, an opposed lower surface 88, a proximal or inlet end 90, and a distal or outlet end 92. The glaucoma device 61 further comprises a plurality of radially outward troughs 64 (64A, 64B, 64C, 64D) and a plurality of radially outward channels 64 (67A, 67B, 67C) to facilitate aqueous transmission or transport. The open troughs 64A, 64B, 64C, 64D and the channels 67A, 67B, and 67C of the device 31 generally provide main passageways for aqueous transmission.

When implanted within the eye 10, the inner space 62 of the stenting glaucoma device 61 is generally in line with the pupil 14 (shown in FIGS. 1 and 2) for light transmission. The body 80 of the device 61 is placed in between the iris 13 and the inner surface 126 (as shown in FIG. 7) of the cornea 12. The inlet end 90 at the inner side 65 is positioned beyond an edge of the dysfunctional anatomic iris closure and the outlet end 92 at the exterior surface 63 is positioned in proximity of the trabecular meshwork 21 of the eye 10.

In some embodiments, the outlet end 92 of the annular member device 61 may further comprise at least one radially protruded construct adapted to be positioned inside an opening of the trabecular meshwork 21. Furthermore, an outlet end of the at least one radially protruded construct may further comprise a trumpet flange adapted for stabilizing the outlet end inside Schlemm's canal 22 of the eye 10 and/or of advantageously promoting outflow characteristics and facilitating in the efficient transport of aqueous through the device 61.

In the illustrated embodiment of FIGS. 7 and 8, and as best seen in FIG. 7, the troughs 64A, 64B, 64C, 64D are formed on the device upper surface 86 and generally radially diverge relative to the central axis 84. As shown in FIG. 7, in some embodiments, one or more troughs 67′ may be provided on the device lower surface 88, as needed or desired. The upper and lower surface troughs may be generally opposed to one another and correspondingly aligned with efficacy, as required or desired, giving due consideration to the goals of providing efficient aqueous transmission and/or of achieving one or more of the benefits as disclosed, taught or suggested herein.

Referring in particular to FIG. 7, the troughs 64A, 64B, 64C, 64D are generally C-shaped or semi-circular. In other embodiments, one or more of the troughs 64A, 64B, 64C, 64D and/or selected portions thereof may be efficaciously shaped in modified manners, as required or desired, giving due consideration to the goals of providing efficient aqueous transmission and/or of achieving one or more of the benefits as disclosed, taught or suggested herein. For example, one or more of the troughs 64A, 64B, 64C, 64D and/or selected portions thereof may be generally U-shaped, V-Shaped, rectangular, semi-elliptical among other suitable polygonal or non-polygonal shapes and combinations thereof. Similarly, one or more of the lower surface troughs 64′ may also be shaped and/or configured as described above for the upper surface troughs 64.

As shown in FIG. 4, the device 61 comprises four upper surface troughs 64A, 64B, 64C, 64D. In another embodiment, the device 61 comprises between two and ten upper surface troughs 64. In yet another embodiment, the device 61 comprises between one and twenty upper surface troughs 64. In other embodiments, the device 61 may efficaciously comprise fewer or more troughs 64, as required or desired, giving due consideration to the goals of providing efficient aqueous transmission and/or of achieving one or more of the benefits as disclosed, taught or suggested herein. Similarly, the number of lower surface troughs 64′ may also be selected as described above for the upper surface troughs 64.

The open troughs 64A, 64B, 64C, 64D (FIG. 7) of the device 61 may be efficaciously arranged on the upper surface 86 in a variety of manners, as required or desired, giving due consideration to the goals of providing efficient aqueous transmission and/or of achieving one or more of the benefits as disclosed, taught or suggested herein. For example, the troughs 64 may be arranged in a generally symmetrical or asymmetrical fashion and/or substantially equidistantly from adjacent troughs 64. In a modified embodiment, one or more of the troughs 64 may interconnect with one or more of the other troughs 64. Similarly, one or more of the lower surface troughs 64′ may also be arranged and/or configured as described above for the upper surface troughs 64.

Referring in particular to FIG. 7, the plurality of channels or lumens 67A, 67B, 67C are formed between the device upper surface 86 and device lower surface 88. The channels 67A, 67B, 67C generally radially diverge relative to the central axis 84. The channels 67A, 67B, 67C have inlet openings or orifices in the device interior surface 65 and outlet openings or orifices in the exterior surface 63.

In the illustrated embodiment of FIGS. 7 and 8, and as best seen in FIG. 7, the channels 67A, 67B, 67C have a generally elliptical or oval shape or cross-section. In another embodiment, one or more selected portions of one or more of the channels 67 may have a generally elliptical or oval shape or cross-section. In other embodiments, selected portions of one or more of the channels 67 may efficaciously be shaped in modified manners, as required or desired, giving due consideration to the goals of providing efficient aqueous transport and/or of achieving one or more of the benefits as disclosed, taught or suggested herein. For example, selected portions of one or more of the channels 67 may have a circular shape or cross-section among other suitable polygonal or non-polygonal shapes or cross-sections and combinations thereof.

In the illustrated embodiment of FIG. 7, the device 61 comprises three channels 67A, 67B, 67C. In another embodiment, the device 61 comprises between two and ten channels 67. In yet another embodiment, the device 61 comprises between one and twenty channels 67. In other embodiments, the device 61 may efficaciously comprise fewer or more channels 67, as required or desired, giving due consideration to the goals of providing efficient aqueous transmission and/or of achieving one or more of the benefits as disclosed, taught or suggested herein.

In the illustrated embodiment of FIG. 7, the channels 67 are arranged such that each channel 67 is below and flanked by a pair of the troughs 64. In this embodiment, the channels 67 are substantially equidistantly arranged such that the spacing between adjacent channels 67 is about the same. In a modified embodiment, one or more of the channels 67 may interconnect with one or more of the other channels 67. In other embodiments, the channels 67 of the device 61 may be efficaciously arranged in a variety of manners, as required or desired, giving due consideration to the goals of providing efficient aqueous transmission and/or of achieving one or more of the benefits as disclosed, taught or suggested herein. For example, the channels 67 may be arranged in a generally symmetrical or asymmetrical fashion, among others.

In some embodiments, the device 61 itself comprises a porous material as has been discussed above in connection with the device 31. One or more selected surfaces of the device 61 may have a plurality of tiny holes or pores for aqueous to diffuse into and out of the device 61 to facilitate efficient transportation of aqueous humor. The holes or pores may provide fluid communication between the aqueous which is exterior to the device 61 and one or more of the device channels 67. The holes or pores may also provide for generally longitudinal flow of aqueous through the device 61.

The glaucoma device 61 (FIGS. 7 and 8) may be made, manufactured or fabricated by a wide variety of techniques. These include, without limitation, molding, thermo-forming, or other micro-machining techniques, among other suitable techniques.

Referring in particular to FIGS. 6 and 7, the glaucoma device 61 is preferably biocompatible so that any inflammation caused by irritation between the outer surface of the device 61 and surrounding tissue is minimal. The device 61 may comprise a biocompatible material, such as medical grade silicone, e.g., Silastic™, available from Dow Corning Corporation of Midland, Mich.; or polyurethane, e.g., Pellethane™, also available from Dow Corning Corporation.

Biocompatible material (biomaterial) suitable for the manufacturing the device 31 may include polyvinyl alcohol, polyvinyl pyrolidone, collagen, heparinized collagen, chemically treated collagen, polytetrafluoroethylene, expanded polytetrafluoroethylene, fluorinated polymer, fluorinated elastomer, flexible fused silica, silicone, polyurethane, poly(methyl methacrylate), acrylic, polyolefin, polyester, polysilicon, polypropylene, hydroxyapetite, titanium, gold, silver, platinum, biodegradable material, bioresorable material, a mixture of two or more of the above biocompatible materials or a mixture of other biocompatible materials, and the like.

In a further embodiment, a composite biocompatible material may be used, wherein a surface material may be used in addition to one or more of the aforementioned materials. Such a surface material may include polytetrafluoroethylene (“PTFE”) (such as Teflon™), polyimide, hydrogel, heparin, hydrophilic coating, therapeutic drugs (such as beta-adrenergic antagonists, other anti-glaucoma drugs, or antibiotics), a combination thereof, and the like.

The glaucoma device of FIGS. 7 and 8 may be efficaciously dimensioned and sized in a variety of manners. The length of the device 61 typically depends on the distance between the anterior chamber 20 and outflow passageways (e.g., trabecular meshwork 21 or a vein) into which the device 61 drains aqueous humor. When the device 61 is placed within the eye 10, the proximal or inlet end 90 is preferably beyond or close to the edge 120 (see FIG. 2) of the iris 13 whereas aqueous is in communication from the anterior chamber 20 into the device 61, as indicated generally by the arrows 122 (FIG. 8). Since in most cases, the trabecular meshwork 21 for angle closure glaucoma is still functional or intact to certain degree, the distal or outlet end 92 may be located at about the inner surface 71 (as shown in FIG. 8) of trabecular meshwork 21 for aqueous transportation using existing outflow pathways, as indicated generally by the arrows 124 (FIG. 8).

In one embodiment, the device 61 has a length of about 5 millimeters (mm). In another embodiment, the device 61 has a length in the range from about 2.5 mm to about 7.5 mm. In yet another embodiment, the device 61 has a length in the range from about 0.5 mm to about 10 mm. Other suitable lengths may also be utilized with efficacy, as needed or desired.

The device 61 also serves to stent the space between the iris 13 and the inner surface 126 (FIG. 8) of the cornea 12. In one embodiment, the device 61 has a thickness of about 250 microns (μm). In another embodiment, the device 61 has a thickness in the range from about of about 200 μm to about 300 μm. In yet another embodiment, the device 61 has a thickness in the range from about 100 μm to about 400 μm. In still another embodiment, the device 61 has a thickness in the range from about 30 μm to about 500 μm. Other suitable thicknesses may also be utilized with efficacy, as needed or desired.

In one embodiment, one or more of the device channels 67 have a diameter or major diameter of about 100 microns (μm). In another embodiment, one or more of the device channels 67 have a diameter or major diameter in the range from about 50 μm to about 200 μm. In yet another embodiment, one or more of the device channels 67 have a diameter or major diameter in the range from about 20 μm to about 250 μm. One or more of the device channels 67 preferably have a minor diameter in the range from about 25% of the channel major diameter to about the same as or about 100% of the major diameter (that is, a circular cross-section). Other suitable channel diameters may also be utilized with efficacy, as needed or desired.

As indicated above, in some embodiments, an opening or perforation in the trabecular meshwork 21 is created for anchoring an inserting section or radially protruding construct of the device 61 inside the trabecular meshwork 21. This opening can be created by laser, a knife, or other surgical cutting instrument. The opening may advantageously be substantially horizontal, i.e., extending longitudinally in the same direction as the circumference of the limbus 15. Other opening directions may also be efficaciously used, such as horizontal or at any angle that is appropriate for inserting the glaucoma device 61 through the trabecular meshwork 21 and into Schlemm's canal or another outflow pathway, as will be apparent to those of skill in the art.

In one embodiment, the method of forming an opening in the trabecular meshwork 21 may comprise making an incision with a microknife, a pointed guidewire, a sharpened applicator, a screw-shaped applicator, an irrigating applicator, or a barbed applicator. Alternatively, or in addition, the trabecular meshwork 21 may be dissected with an instrument similar to a retinal pick, or a microcurrette. In another embodiment, the opening may be created by fiberoptic laser ablation. In one preferred embodiment, a device delivery applicator comprising an opening-creating capability is used to facilitate creating an opening in the trabecular meshwork 21 and inserting the glaucoma device 61 in one operating procedure.

A further aspect of the invention includes methods for increasing aqueous humor outflow in an eye 10 of a patient to reduce intraocular pressure therein. One method involves placing the glaucoma device 61 into the anterior chamber 20 of the eye 10 for reducing intraocular pressure in a patient having a dysfunctional anatomical iris closure in angle closure glaucoma. The method generally comprises advancing the glaucoma device 61 using a delivery applicator through an incision of the eye 10 and positioning the device 61 at about the dysfunctional anatomical iris closure. Then aqueous humor is transmitted through the device 61 and enters the trabecular meshwork 21, from the deep side to the superficial side of the trabecular meshwork 21. This “transmitting” of aqueous humor is, in one aspect of the invention, preferably passive, i.e., aqueous humor is allowed to flow out of the anterior chamber 20 due to the pressure gradient between the anterior chamber 20 and the aqueous venous system including the aqueous veins 23.

The glaucoma device 61 of FIGS. 7 and 8 can be placed at the implantation site in a manner similar to the description above in connection with the glaucoma device 31 and FIG. 6. Referring back to FIG. 6 an irrigating knife or device delivery applicator 51 is provided, which, in some embodiments, comprises a syringe portion 54 and a cannula portion 55. The cannula portion 55 may be curved to facilitate inserting the device 61 into the anatomical iris closure. The distal section of the cannula portion 55 has at least one optional irrigating hole 53 and a distal space 56 for holding the device 61. The proximal end 57 of the lumen of the distal space 56 is, in one embodiment, sealed off from, and thus substantially not in communication with, the remaining lumen of the cannula portion 55. In this embodiment, the device 61 is placed on the delivery applicator 51 and advanced to the implant site, wherein the delivery applicator 51 holds the device 61 securely during delivery and releases it when the surgeon chooses to deploy the device 61. An optional cutting knife at the distal end of the applicator 51 renders the two steps of slitting and device deployment in one operating procedure.

In some embodiments of trabecular meshwork surgery in accordance with the invention, the patient is placed in the supine position, prepped, draped, and anesthetized as necessary. In one embodiment, a small (typically less than about 1 mm) incision 52 (see FIG. 6), which may be self-sealing, is made through the cornea 12. Through this incision, the trabecular meshwork 21 is accessed, and an incision is made in the trabecular meshwork 21 with an irrigating knife. The device 61 is then advanced through the corneal incision 52 across the anterior chamber 20, while the device 61 is held in an irrigating applicator 51, under gonioseopic, microscopic, or endoscopic guidance. After the device 61 is implanted in place, the applicator 51 is withdrawn and the surgery concluded. The irrigating knife may be within a size range of about 16 to about 40 gauge, and, in some embodiments, preferably about 30 gauge.

It is one preferred embodiment that the annular device 61 is placed, anchored, or implanted inside the anterior chamber 20 so that adequate aqueous humor is transported from the anterior chamber 20 through tissue of the trabecular meshwork 21 to enter Schlemm's canal 22, which then empties into aqueous collector channels in the posterior wall of Schlemm's canal 22 and then into aqueous veins 23 (see FIG. 2), which form the episcleral venous system.

As indicated above, the glaucoma device 61 when implanted inside the anterior chamber 20 has the inlet end 90 positioned beyond or close to an edge of the dysfunctional anatomic iris closure and the outlet end 92 is positioned in proximity of the trabecular meshwork 21 of the eye 10.

As also indicated above, FIG. 8 shows a perspective view of the anterior chamber 20 of the eye 10. The drawing illustrates the glaucoma device 61 positioned inbetween the iris 13 and the cornea 12 to stent dysfunctional anatomical iris closure. The outlet end 92 of the exterior surface 63 of the device 61 lies close to trabecular meshwork 21 which is functional in this case.

From the foregoing description, it will be appreciated that a novel approach for the surgical treatment of angle closure glaucoma has been disclosed. While the components, techniques and aspects of the invention have been described with a certain degree of particularity, it is manifest that many changes may be made in the specific designs, constructions and methodology herein above described without departing from the spirit and scope of this disclosure.

Various modifications and applications of the invention may occur to those who are skilled in the art, without departing from the true spirit or scope of the invention. It should be understood that the invention is not limited to the embodiments set forth herein for purposes of exemplification, but is to be defined only by a fair reading of the appended claims, including the full range of equivalency to which each element thereof is entitled.

What is claimed is:

1. A glaucoma device for reducing intraocular pressure in a patient having angle closure glaucoma, the glaucoma device comprising an aqueous transporting element for transporting aqueous humor to bypass dysfunctional anatomical iris closure and restoring existing outflow pathways of said anatomical iris closure, the aqueous transporting element having an inlet end and an outlet end, wherein the inlet end is positioned inside an anterior chamber of an eye beyond an edge of said dysfunctional anatomic iris closure and the outlet end is positioned at proximity of trabecular meshwork of the eye.

2. The glaucoma device according to claim 1, wherein said aqueous transporting element is an elongate tubular member.

3. The glaucoma device according to claim 2, wherein the elongate tubular member further comprises an inlet section having said inlet end and an outlet section having said outlet end, the inlet section being at an angle in relation to the outlet section.

4. The glaucoma device according to claim 3, wherein the outlet section is positioned at an opening of trabecular meshwork, the opening being created by incision or perforation.

5. The glaucoma device according to claim 1, wherein the device is made of a biocompatible material selected from a group consisting of polyvinyl alcohol, polyvinyl pyrolidone, collagen, heparinized collagen, chemically treated collagen, polytetrafluoroethylene, expanded polytetrafluoroethylene, fluorinated polymer, fluorinated elastomer, flexible fused silica, silicone, polyurethane, poly(methyl methacrylate), acrylic, polyolefin, polyester, polysilicon, polypropylene, hydroxyapetite, titanium, gold, silver, platinum, biodegradable material, bioresorable material, and a mixture thereof.

6. The glaucoma device according to claim 5, wherein the biocompatible material comprises surface coating with a coating material selected from the group consisting of Teflon, polyimide, hydrogel, heparin, hydrophilic coating substrate, therapeutic drug, and a combination thereof.

7. The glaucoma device according to claim 2, wherein the device is made of a porous material.

8. The glaucoma device according to claim 2, wherein the device is made of a solid material with many interconnected tiny holes for communicating aqueous humor throughout said interconnected holes.

9. The glaucoma device according to claim 4, wherein the outlet end further comprises a trumpet flange adapted for stabilizing the outlet end inside Schlemm's canal of the eye.

10. The glaucoma device according to claim 7 or claim 8, the device further comprising at least an elongate trough for transmitting aqueous humor between the inlet end and the outlet end of the glaucoma device.

11. The glaucoma device according to claim 10, wherein said at least one elongate trough is in communication with a lumen of the glaucoma device between the inlet end and the outlet end.

12. The glaucoma device according to claim 3, wherein the angle is between about 120 degrees to about 185 degrees.

13. The glaucoma device according to claim 1, wherein said aqueous transporting element is an annular member, the annular member being placed inside the anterior chamber, wherein the inlet end is positioned beyond an edge of said dysfunctional anatomic iris closure and the outlet end is positioned at proximity of trabecular meshwork of the eye.

14. The glaucoma device according to claim 13, wherein the annular member is selected from the group consisting of a ring, an oval ring, and a semi-open ring configured to fit inside the anterior chamber of the eye.

15. The glaucoma device according to claim 14, wherein the outlet end of said annular member further comprises at least one protruded construct adapted to be positioned inside an opening of trabecular meshwork, the opening being created by incision or perforation.

16. The glaucoma device according to claim 13 or claim 15, wherein the device is made of a biocompatible material selected from a group consisting of polyvinyl alcohol, polyvinyl pyrolidone, collagen, heparinized collagen, chemically treated collagen, polytetrafluoroethylene, expanded polytetrafluoroethylene, fluorinated polymer, fluorinated elastomer, flexible fused silica, silicone, polyurethane, poly(methyl methacrylate), acrylic, polyolefin, polyester, polysilicon, biodegradable material, bioresorable material, and a mixture thereof.

17. The glaucoma device according to claim 16, wherein the biocompatible material comprises surface coating with a coating material selected from a group consisting of Teflon, polyimide, hydrogel, heparin, hydrophilic coating substrate, therapeutic drug, and a combination thereof.

18. The glaucoma device according to claim 13 or claim 15, wherein the device is made of a porous material.

19. The glaucoma device according to claim 13 or claim 15, wherein the device is made of a solid material with many interconnected tiny holes for communicating aqueous humor throughout said interconnected holes.

20. The glaucoma device according to claim 15, wherein an outlet end of the at least one protruded construct further comprises a trumpet flange adapted for stabilizing the outlet end within Schlemm's canal of the eye.

21. The glaucoma device according to claim 18 or claim 19, the device further comprising at least an elongate trough for transmitting aqueous humor between the inlet end and the outlet end of the glaucoma device.

22. The glaucoma device according to claim 20, wherein said at least one elongate trough is in communication with a lumen of the glaucoma device between the inlet end and the outlet end.

23. A method of placing a glaucoma device into an anterior chamber of an eye for reducing intraocular pressure in a patient having a dysfunctional anatomical iris closure in angle closure glaucoma, the method comprising advancing said glaucoma device over a delivery device through an incision of the eye and positioning said device at about said dysfunctional anatomical iris closure for restoring normal aqueous flow inside the eye.

24. The method according to claim 23, wherein the glaucoma device comprises an elongate tubular member for transporting aqueous humor to bypass dysfunctional anatomical iris closure and restoring existing outflow pathways of said anatomical iris closure, the elongate tubular member having an inlet end and an outlet end, wherein the inlet end is positioned inside an anterior chamber of an eye beyond an edge of said dysfunctional anatomic iris closure and the outlet end is positioned at proximity of trabecular meshwork of the eye.

25. The method according to claim 24, the method further comprising positioning the outlet section at an opening of trabecular meshwork, the opening being created by incision or perforation.

26. The method according to claim 23, wherein the glaucoma device comprises an annular member having an inlet end and an outlet end, wherein the annular member is positioned inside the anterior chamber, and wherein the inlet end is positioned beyond an edge of said dysfunctional anatomic iris closure and the outlet end is positioned at proximity of trabecular meshwork of the eye.

27. A method for reducing intraocular pressure in a patient having a dysfunctional anatomical iris closure in angle closure glaucoma, the method comprising placing a glaucoma device having an aqueous transporting element for transporting aqueous humor to bypass dysfunctional anatomical iris closure and restoring existing outflow pathways of said anatomical iris closure at about said dysfunctional anatomical iris closure.

28. The method of claim 27, wherein the step of placing the glaucoma device is an ab interno procedure.
